(19) United States
(12) Patent Application Publication
(10) Pub. No.: US 2026/0279517 A1
(43) Pub. Date: Sep. 17, 2026

(54) GENERATING A CLINICAL TRIAL DATABASE BY USING COMPUTER-EXECUTABLE INSTRUCTIONS TO INTERACT WITH A GRAPHICAL USER INTERFACE OF A THIRD-PARTY DATA COLLECTION SYSTEM

(71) Applicant: Pentara, Inc., Millcreek, UT (US)

(72) Inventors: David Steinberg, Taylorsville, UT (US); Brandon Adkinson, Waukesha, WI (US); Kent Hendrix, Millcreek, UT (US)

(21) Appl. No.: 19/081,974

(22) Filed: Mar. 17, 2025

(51) Int. Cl. G16H 10/20

(52) U.S. Cl. CPC G16H 10/20

(57) ABSTRACT

The present disclosure relates to systems, non-transitory computer-readable media, and methods for generating a clinical trial database by utilizing computer-executable instructions to interact with a graphical user interface of a third-party data collection system and perform interactions based on clinical trial templates and a clinical trial standard. In particular, in one or more embodiments, the disclosed systems select clinical trial templates, and a clinical trial standard based on configuration data. The disclosed systems then utilize computer-executable instructions to identify and update elements of a graphical user interface of a third-party data collection system based on the clinical trial template and the clinical trial standard.

### BACKGROUND

In recent years, significant improvements have been made in utilizing digital databases for collecting and storing data for clinical trials, streamlining clinical trials by enhancing data accessibility and real-time insights. For example, conventional digital databases are often cloud-based, which enables remote teams to access and update clinical data from virtually any location, ensuring collaboration and access to data between providers, patients, investigators, and sponsors without the need for physical proximity. In addition, because all participants of a clinical trial access the same digital database to enter clinical data during the clinical trial, conventional digital databases ensure consistency in the collected clinical data. However, though conventional digital database systems offer increased ease of use and consistency of clinical data, a number of problems and issues arise in generating a clinical trial database.

For instance, conventional digital database systems require excessive user interactions to generate a digital database for a clinical trial. Specifically, conventional digital database systems render a graphical user interface with various elements, with elements corresponding to establishing variables, participant information, protocols, visits, and various other information for portions of the digital database into which administrators of a clinical trial will enter data. In order to establish the digital database, conventional digital database systems require a user to interact with multiple elements across multiple graphical user interfaces, entering different variables and specifications for the clinical trial. In some cases, elements contain multiple options that each require interactions to generate a specific portion of the digital database

In part due to the excessive user interactions, conventional digital database systems also generate inaccurate digital databases and, as a result, inaccurate clinical trial results. Specifically, because conventional digital database systems require human interaction to generate a digital database, simple human error results in errors or omissions of the digital database for the clinical trial, leading to missing data collection points or inconsistent usage of variables. Indeed, as a human attempts to organize and access multiple data points for information to include in a clinical trial database, a human may enter data differently (or incorrectly), omit variables, or simply misspell items so they don't match. In addition, conventional digital database systems fail to account for clinical standards upon generating the digital database and instead retrofit the clinical trial standards into a previously generated digital database, leading to additional errors and inconsistencies. Moreover, as a digital database can potentially be accessed by hundreds of administrators of a clinical trial, even a single error in a digital database can result in massive deviations in the accuracy of clinical trial data.

In addition, conventional digital database systems require excessive usage of computing resources. For example, conventional digital database systems require computers to spend excessive amounts of time rendering graphical user interfaces, performing operations, and excessively processing calls as a human enters each data point, variable, instrument, standard, or other piece of information for the clinical trial. In addition, as users need to identify various pieces of data and/or information, users often render multiple graphical user interfaces and move between them, requiring excessive processing and computing power.

In addition, generating a digital database for a clinical trial leads to unique problems. For instance, clinical trials are required to adhere to multiple standards, regulations, and other government requirements for a regulatory or government agency to approve the clinical trial, including clinical trial standards, frameworks, and guidelines that standardize the collection and organization of clinical data collected from clinical trials. Third-party data collection systems can include the necessary components to generate clinical trial databases, such as graphical user interfaces, which include the necessary options and elements for the required variables and clinical trial standards. However, third-party data collection systems still require manual input of variables, clinical standards, and other information to build a clinical trial database for a clinical trial. Moreover, third-party data collection systems do not provide options for generating additional documentation that is also required to approve a clinical trial database, such as validation documentation. These, along with additional problems and issues, exist with regard to conventional clinical trial systems.

### SUMMARY

Embodiments of the present disclosure provide benefits and/or solve one or more of the foregoing or other problems in the art with systems, non-transitory computer-readable media, and methods for generating a clinical trial database by using computer-executable instructions to interact with a graphical user interface of a third-party data collection system based on clinical trial templates and a clinical trial standard. For example, in one or more embodiments, the disclosed systems select clinical trial templates and a clinical trial standard for the clinical trial based on specifications indicated in configuration data. The disclosed systems then utilize computer-executable instructions to interact with a third-party data collection system and generate the clinical trial database. In some cases, the disclosed systems utilize computer-executable instructions to identify and update elements of graphical user interfaces of the third-party data collection system using the clinical trial template or the clinical trial standard. Additional features and advantages of one or more embodiments of the present disclosure are outlined in the description that follows and, in part, will be obvious from the description or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an example diagram of an overview of a clinical trial database generation system in accordance with one or more embodiments.

FIG. 2 illustrates a schematic diagram of a clinical trial database generation system utilizing computer-executable instructions to interact with a graphical user interface of a third-party data collection system as indicated in clinical trial templates and consistent with a clinical trial standard in accordance with one or more embodiments.

FIG. 3 illustrates a schematic diagram of a clinical trial database generation system utilizing a data-identifying machine-learning model to extract clinical trial specifications and a clinical trial standard version from configuration data in accordance with one or more embodiments.

FIG. 4 illustrates a schematic diagram of a clinical trial database generation system generating clinical trial requirements in accordance with one or more embodiments.

FIG. 5 illustrates an example graphical user interface of a configuration graphical user interface of a clinical trial database generation system for receiving configuration data in accordance with one or more embodiments.

FIG. 6 illustrates an example graphical user interface of a clinical trial database generation system utilizing computer-executable instructions to interact with elements of a graphical user interface based on corresponding clinical trial template(s) or a clinical trial standard in accordance with one or more embodiments.

FIG. 7 illustrates an example graphical user interface of a clinical trial database generation system utilizing computer-executable instructions to make selections within an element in accordance with one or more embodiments.

FIGS. 8A-8C illustrates an example graphical user interface of a clinical trial database generation system interacting with and modifying an element in accordance with one or more embodiments.

FIG. 9 illustrates a diagram of an environment in which a clinical trial database generation system can operate in accordance with one or more embodiments.

FIG. 10 illustrates a flowchart of a series of acts for utilizing computer-executable instructions to generate a clinical database based on clinical trial templates and clinical trial standards in accordance with one or more embodiments.

FIG. 11 illustrates a block diagram of an example computing device for implementing one or more embodiments of the present disclosure.

FIG. 12 illustrates a network of a clinical data management system in accordance with one or more embodiments.

### DETAILED DESCRIPTION

This disclosure describes one or more embodiments of a clinical trial database generation system that generates a clinical trial database by utilizing computer-executable instructions to interact with a third-party data collection system using clinical trial templates and clinical trial standards. For example, the clinical trial database generation system selects one or more clinical trial templates and a clinical trial standard based on the configuration data. The clinical trial database generation system then utilizes computer-executable instructions to interact with graphical user interfaces of a third-party data collection system using the clinical trial templates and the selected clinical trial standard. In some instances, the clinical trial database generation system identifies elements of graphical user interfaces of the third-party data collection system and updates the elements using data from the clinical trial templates or the clinical trial standard.

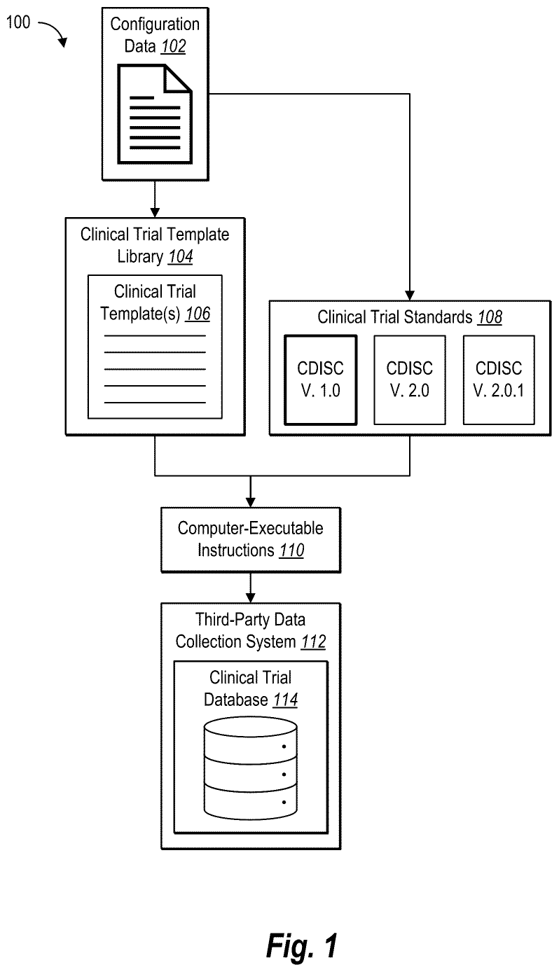

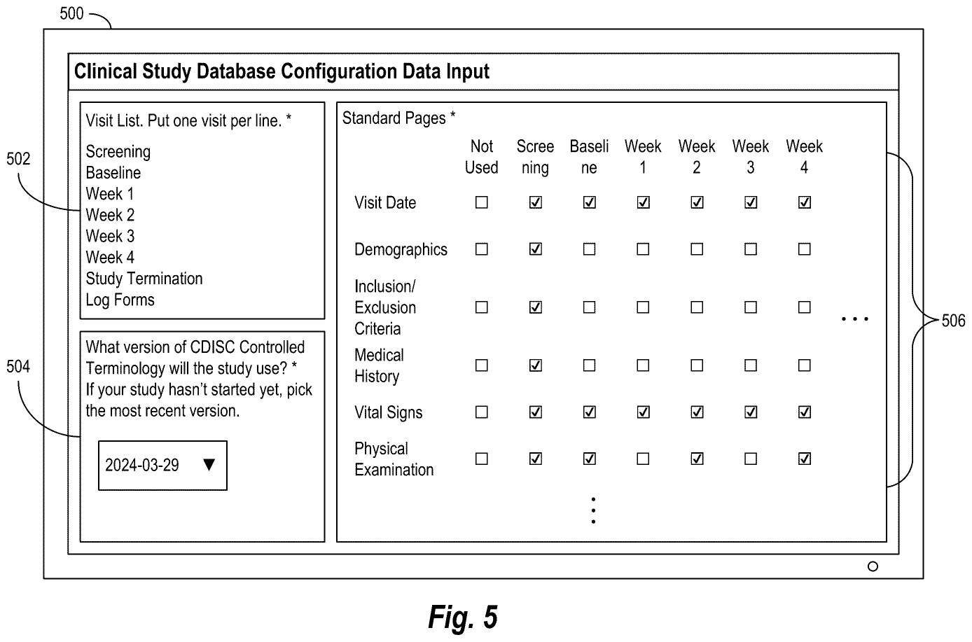

FIG. 1 illustrates an example overview of clinical trial database generation system 100 utilizing the computer-executable instructions to generate a clinical trial database. As shown, the clinical trial database generation system 100 receives configuration data 102. Specifically, the clinical trial database generation system 100 receives configuration data 102 that indicates the specifications (or design) of a clinical trial. For example, configuration data 102 can indicate visits (e.g., clinical visits) for the clinical trial, data that will be collected during the visits, and a version of clinical standard to which the clinical trial will adhere. Additional details and examples of the clinical trial database generation system 100 receiving configuration data are provided with respect to FIG. 5 below.

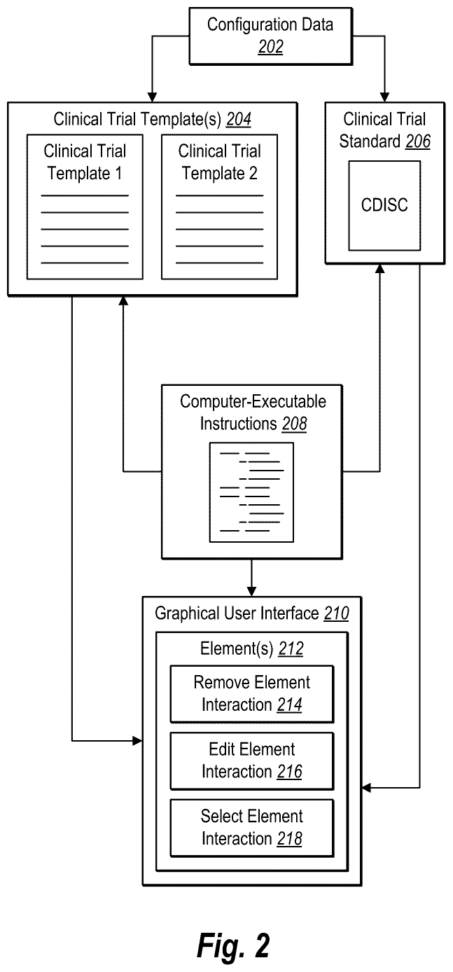

As also shown, the clinical trial database generation system 100 selects clinical trial template(s) 106 based on configuration data 102. Specifically, based on specifications for a clinical trial indicated in configuration data 102, the clinical trial database generation system 100 selects one or more clinical trial template(s) 106 from the clinical trial template library 104. For example, the clinical trial database generation system 100 (or the clinical data management system 904) maintains a library (or database) of clinical trial templates that correspond to various types of clinical trials. The clinical trial database generation system 100 then selects clinical trial template(s) 106 for a clinical trial based on configuration data 102. In some cases, clinical trial template(s) 106 corresponds to data that will be collected during the clinical trial and/or the status of the patient, such as whether data is being collected to qualify a patient for a clinical trial or whether data is being collected during a clinical trial. Additional details regarding the clinical trial database generation system 100 utilizing clinical trial template(s) 106 are provided with respect to FIG. 2 below.

In addition, as shown, the clinical trial database generation system 100 selects a clinical trial standard from clinical trial standards 108. Specifically, clinical trial standards 108 are standards (or guidelines) and corresponding data formats that guide the collection, organization, analysis, and submission of clinical data of a clinical trial. For example, regulatory authorities or other governing bodies that oversee clinical trials (e.g., the U.S. Food and Drug Administration or the European Medicines Agency) require or highly recommend clinical trials adhere to certain clinical trial standards. In some cases, clinical trial standards 108 are standards developed and maintained by the Clinical Data Interchange Standards Consortium (CDISC) and/or the NIH Cancer Institute.

In one or more embodiments, as shown, the clinical trial database generation system 100 selects a clinical trial standard based on configuration data 102. Specifically, the clinical trial database generation system 100 identifies a clinical trial standard version indicated in configuration data 102 and selects a corresponding clinical trial standard from clinical trial standards 108. For example, if configuration data 102 indicates that the clinical trial will use CDISC version 1.0, then the clinical trial database generation system 100 will select that clinical trial standard for the clinical trial. Additional details regarding the clinical trial database generation system 100 selecting a clinical trial standard are provided with respect to FIG. 2 below.

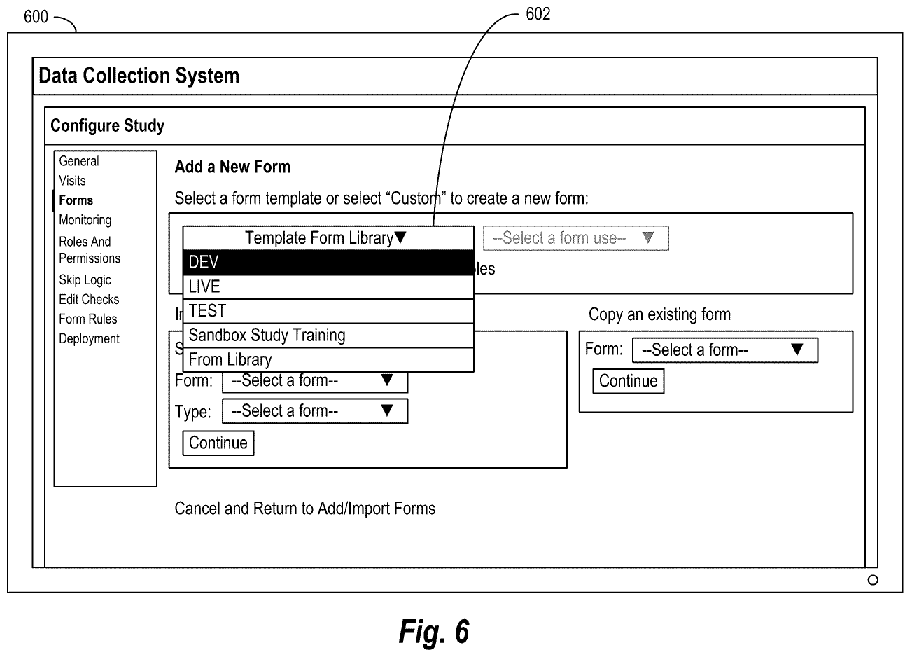

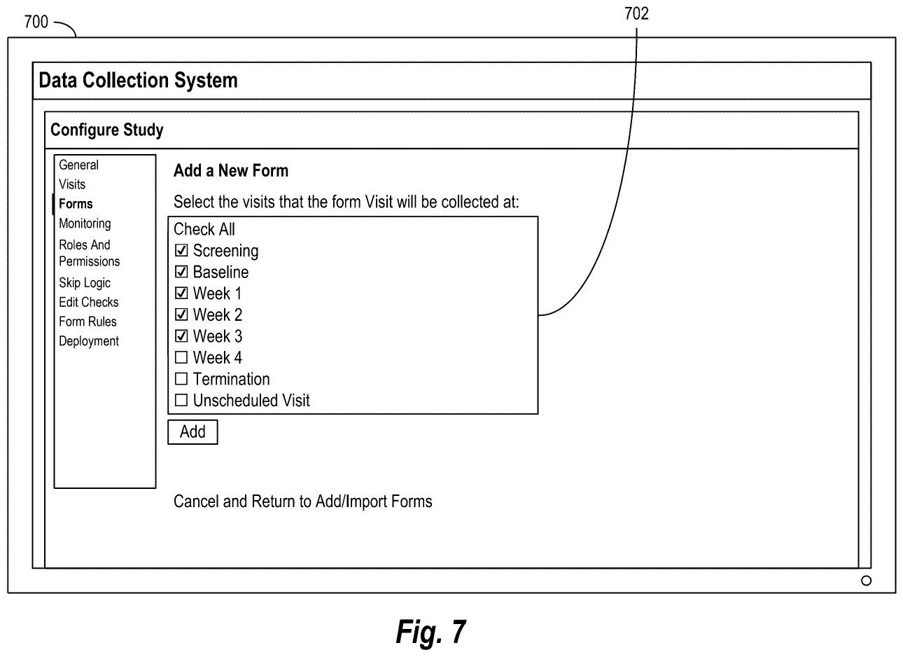

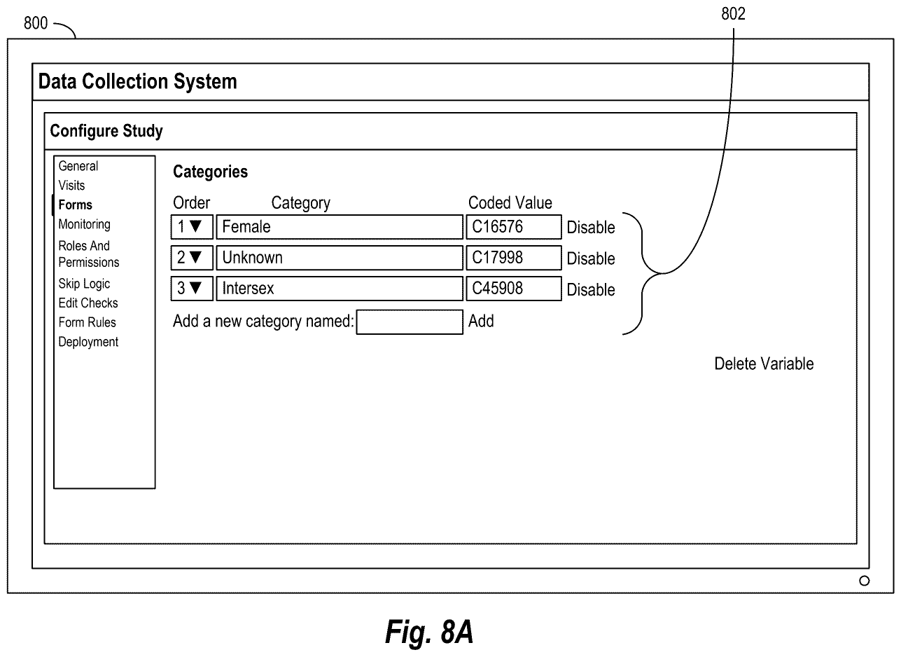

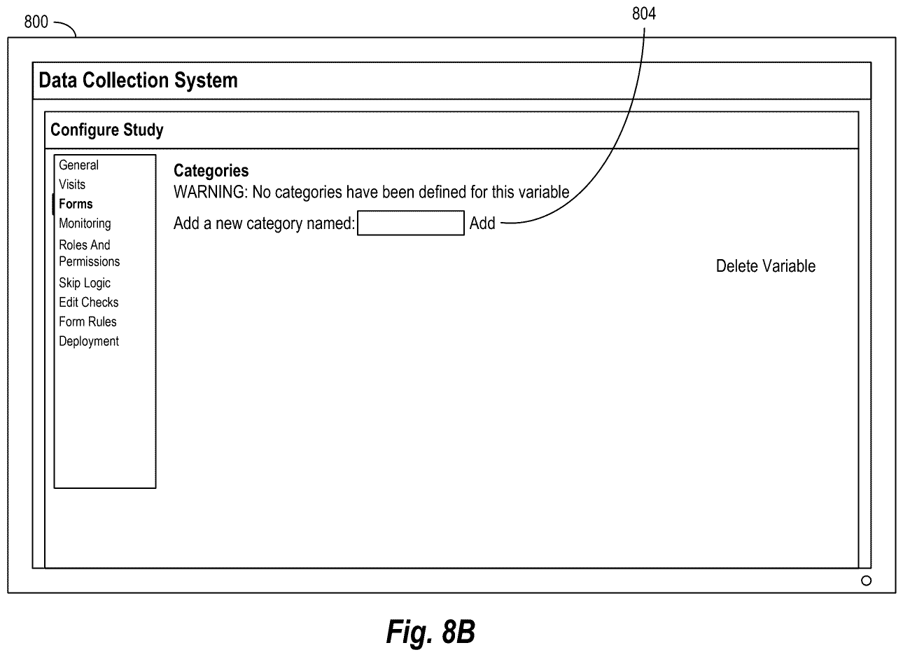

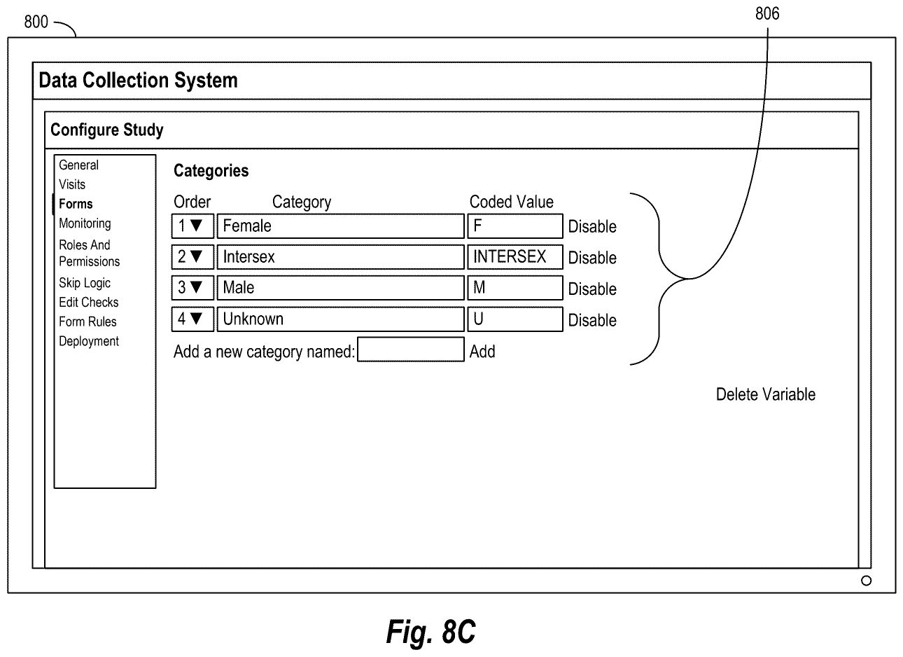

As also shown, the clinical trial database generation system 100 utilizes computer-executable instructions 110 to interact with third-party data collection system 112 and generate clinical trial database 114. Specifically, computer-executable instructions 110 interact with a graphical user interface of third-party data collection system 112 to identify and update elements of the graphical user interface to reflect data from clinical trial template(s) 106 and/or the clinical trial standard. For example, computer-executable instructions 110 edit an element, identify an element, select an element, or instruct the third-party data collection system 112 to remove elements. Additional details regarding the clinical trial database generation system 100 utilizing computer-executable instructions to interact with elements of a graphical user interface of a third-party data collection system are provided with respect to FIG. 2 below. Additionally, examples of the clinical trial database generation system 100 interacting with elements of a graphical user interface of a third-party data collection system are provided with respect to FIG. 6, FIG. 7. and FIGS. 8A-8C below.

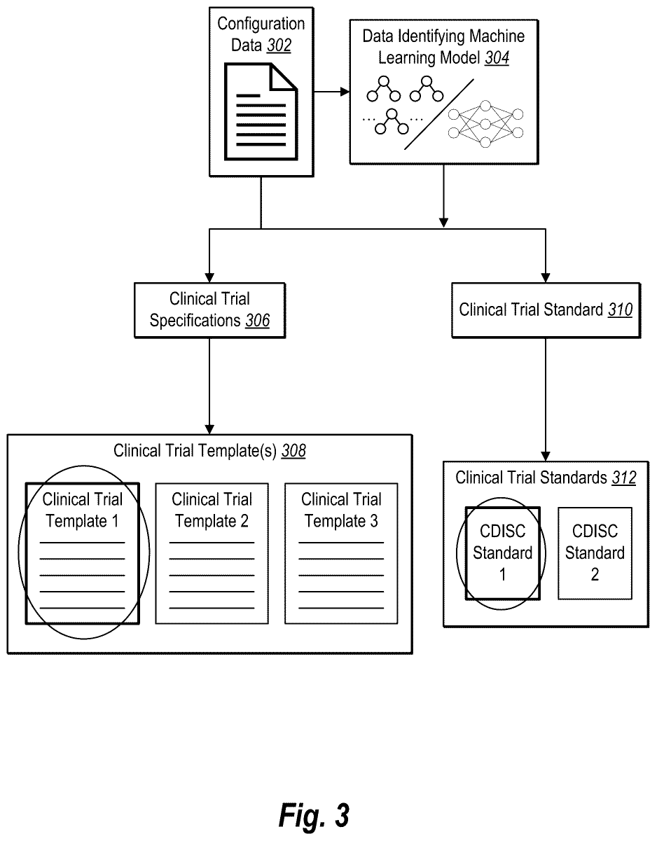

As mentioned, the clinical trial database generation system 100 utilizes configuration data 102 to select clinical trial template(s) 106 and selects a clinical trial standard from clinical trial standards108 based on a clinical trial standard indicated in configuration data 102. In addition, in one or more embodiments, the clinical trial database generation system 100 utilizes a data-identifying machine-learning model to process configuration data to process configuration data 102 and identify specifications and/or clinical trial standards found within configuration data 102. Additional details regarding the clinical trial database generation system 100 utilizing a data-identifying machine-learning model to process configuration data are provided with respect to FIG. 3 below.

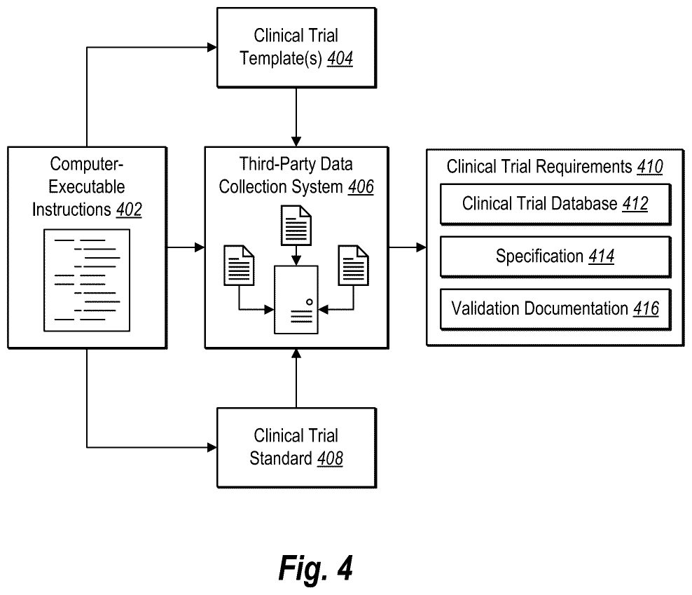

In one or more embodiments, the clinical trial database generation system 100 generates requirements for a clinical trial in addition to a clinical trial database 114. Specifically, the clinical trial database generation system 100 generates additional documents and forms required by regulatory agencies for a clinical trial. For example, the clinical trial database generation system 100 can generate a specification and a validation document in addition to a clinical database for a clinical trial. Additional details regarding the clinical trial database generation system 100 generating additional clinical trial requirements are provided with respect to FIG. 4 below.

The clinical trial database generation system 100 provides a variety of technical advantages relative to conventional systems. For example, by utilizing computer-executable instructions to interact with a graphical user interface of a third-party data collection system, the clinical trial database generation system 100 reduces the number of user interface interactions required by conventional digital database systems. Specifically, the clinical trial database generation system 100 utilizes computer-executable instructions that operate autonomously within a graphical user interface, enabling selections and generating a clinical trial database without user interaction (or minimal user interactions to initiate the computer-executable instructions).

In addition, by utilizing computer-executable instructions to enter data based on clinical trial templates and/or clinical trial standards, the clinical trial database generation system 100 improves accuracy relative to conventional digital database systems. For example, computer-executable instructions include instructions for how to interact with various graphical user interfaces of a third-party data collection system and clinical trial template(s) from which to pull data. Moreover, computer-executable instructions also include instructions to pull data from a clinical trial standard that is established for the clinical trial in configuration data, ensuring that the clinical trial database generation system 100 generates a clinical trial database that utilizes consistent standards throughout the database. Indeed, because computer-executable instructions are working autonomously and pulling data from pre-made clinical trial templates, the clinical trial database generation system 100 doesn't suffer from the same manual input errors that plague conventional digital database system.

Additionally, by decreasing the need for manual input of all information, the clinical trial database generation system 100 increases efficiency over conventional digital database systems. Specifically, by utilizing computer-executable instructions to interact with a graphical user interface of a third-party data collection system, the clinical trial database generation system 100 can generate a clinical trial database in a fraction of time required by conventional digital database systems and thus require less computing and processing power. Moreover, by utilizing clinical trial templates that work with the computer-executable instructions for multiple different kinds of clinical trials, the clinical trial database generation system 100 further reduces the processing and computing power to manually enter information for each clinical trial database.

Moreover, the clinical trial database generation system 100 solves problems that are unique to clinical trial databases. For example, by using computer-executable instructions that can interact with a variety of clinical trial templates, the clinical trial database generation system 100 generates clinical trial databases that adhere to certain standards and utilize consistent variables and naming standards in a fraction of the time that it takes for conventional digital database systems. To illustrate, while it would generally take several hours to manually enter information into a conventional digital database system, the clinical trial database generation system 100 can generate a clinical trial database, a specification, and validation documentation in under an hour—even a matter of minutes. Further, the clinical trial database generation system 100 generates additional clinical trial requirements, such as a specification and validation documentation, as it generates the clinical trial database, ensuring consistency across all documents, an extremely difficult task when generating this documentation manually and separately from the clinical trial database.

As illustrated by the foregoing discussion, the present disclosure utilizes a variety of terms to describe the features and advantages of the clinical trial database generation system. Additional details regarding the meaning of such terms are now provided. For example, as used herein, the term “clinical trial database” can include data and related storage components that relate to a clinical trial. In particular, the term “clinical trial database” refers to a centralized repository that collects, organizes, and stores data and other information relating to a clinical trial. For example, a clinical trial database can include a database that is tailored for access and data entry for collecting data for the clinical trial.

In addition, as used herein, the term “configuration data” can include data for setting up a clinical trial. Specifically, the term “configuration data” can include foundational information gathered and documented during the planning and setup phase of a clinical trial and provides the framework for the operational execution of the clinical trial. For example, configuration data can include details and specifications for the structure of a clinical trial or for managing the clinical trial. To illustrate, configuration data can include protocol information, visit schedules, data collection plans, eligibility criteria, site information (e.g., clinical trial sites), informed consent processes, endpoints and outcomes, and monitoring and reporting.

Also, as used herein, the term “clinical trial template” refers to a framework or structure that serves as a starting point for a clinical trial. Specifically, the term “clinical trial template” refers to a standardized framework or structure that outlines the key components, structure, and requirements needed to design, document, and implement a clinical trial. In some cases, a clinical trial template can refer to a standardized framework for a specific type of clinical trial. For example, a clinical trial template can include a framework used for testing corresponding to a specific illness (e.g., Alzheimer's Disease or Parkinson's Disease). Relatedly, as used herein, the term “template data” refers to data from a clinical trial template. For example, “template data” can refer to data or information accessed from a clinical trial template.

Moreover, as used herein, the term “clinical trial standard” refers to guidelines that standardize the collection, organization, and exchange of data for clinical studies. In particular, the term “clinical trial standard” refers to mandated guidelines for study design, data collection, data tabulation, data analysis, and terminology for a clinical trial. In some cases, a clinical trial standard refers to a Clinical Data Interchange Standards Consortium (“CDISC”) standard. For example, a clinical trial standard can refer to a specific version of a CDISC standard. Relatedly, the term “standard data” refers to data from a clinical trial standard. For example, “standard data” can refer to data or information accessed from a clinical trial standard.

As used herein, the term “third-party data collection system” refers to a system to systematically gather, organize, and store clinical data for a clinical study. Specifically, the term “third-party data collection system” refers to a digital platform or electronic database that can be used to set up a clinical trial and also used for collecting data during a testing portion of a clinical trial. For example, the term third-party data collection system refers to a system that can be accessed by various client devices across various locations to input clinical data (e.g., testing data) during a clinical trial. In some cases, the term third-party data collection system refers to an electronic database that is located externally to (e.g., separate from) a clinical trial database generation system or a clinical data management system

As previously mentioned, the clinical trial database generation system 100 can utilize computer-executable instructions to interact with a third-party data collection system. Specifically, in one or more embodiments, the clinical trial database generation system 100 utilizes computer-executable instructions to interact with elements of a graphical user interface of a third-party data collection system and add data from clinical trial templates and a clinical trial standard. FIG. 2 illustrates a schematic diagram of a clinical trial database generation system utilizing computer-executable instructions to interact with a graphical user interface of a third-party data collection system as indicated in clinical trial templates and consistent with a clinical trial standard in accordance with one or more embodiments.

As shown in FIG. 2, the clinical trial database generation system 100 selects clinical trial template(s) 204 based on configuration data 202. In one or more embodiments, the clinical trial database generation system 100 receives configuration data 202 that indicates specifications for a clinical trial and selects clinical trial template(s) 204 that correspond to the specifications of the clinical trial. Specifically, based on indications of steps, stages, or a setup of the clinical trial within the configuration data, the clinical trial database generation system 100 selects clinical trial template(s) 204 for generating a clinical database that implements various steps or stages of the clinical trial. For example, if configuration data 202 indicates that the clinical trial will include a clinical visit where clinical staff will conduct a specific screening (e.g., MMSE or ADAS-Cog), the clinical trial database generation system 100 selects a clinical trial template from clinical trial template(s) 204 corresponding to the specific screening and the clinical trial database generation system 100 utilizes the selected clinical trial template to generate the clinical database with the specified screening.

In some cases, clinical trial template(s) 204 includes template data that corresponds to the type of clinical data collected during a clinical trial. Specifically, template data corresponds to a specific format for collecting data during a clinical trial. For example, a clinical trial template can include template data for an instrument used during the clinical trial to collect clinical data. To illustrate, if a clinical trial is going to use an MMSE screening for an initial visit, a clinical trial template for an MMSE screening can include template data for including an MMSE screening in a clinical trial. In addition, the clinical trial database generation system 100 can maintain clinical trial templates that relate to clinical trial specifications for a certain type of clinical trial (e.g., for a drug for treating a certain type of disease). For instance, the clinical trial database generation system 100 can maintain clinical trial templates that contain template data for frequent instruments for testing drugs for Alzheimer's disease.

As previously mentioned, in one or more embodiments, the clinical trial database generation system 100 (or the clinical data management system 904) maintains a clinical trial template library of clinical trial templates, and the clinical trial database generation system 100 selects clinical trial templates from the clinical trial template library. Specifically, the clinical trial database generation system 100 maintains a clinical trial template library of clinical trial templates that correspond to different types of clinical data collected in a clinical trial. Based on the clinical trial specifications in the configuration data, the clinical trial database generation system 100 selects clinical trial template(s) 204 from the clinical trial template library.

As also shown, the clinical trial database generation system 100 selects a clinical trial standard 206. In particular, the clinical trial database generation system 100 selects a clinical trial standard based on a clinical trial standard version indicated in configuration data 202. For example, a regulatory agency or governing body that oversees clinical trial standards has several versions of clinical trial standards, and each clinical trial standard version contains different versions of standard data for clinical trials. To illustrate, different clinical trial standard versions can include different demographic data, such as race. One clinical trial standard version could include Black, White, Hispanic, or Other, while another clinical trial standard version includes American Indian or Alaska Native, Asian, Black or African American, Native Hawaiian or Other Pacific Islander, White, Not Reported, Unknown, or Other.

In one or more embodiments, the clinical trial template(s) 204 are aligned to clinical trial standard 206. Specifically, the clinical trial templates are designed so that the structure of the templates and, therefore, the clinical trial database is aligned with a clinical trial standard. For example, a clinical trial template can be formatted to use vertically or horizontally transposed data to match a clinical trial standard. As another example, variables in clinical trial templates correspond to variables in clinical trial standards. Indeed, by designing the clinical trial templates so they align with the clinical trial standards, the clinical trial database generation system 100 is able to more accurately and efficiently generate clinical trial databases and other components of a clinical trial than conventional clinical trial setup systems.

In addition, in one or more embodiments, clinical trial template(s) 204 include skip logic. In particular, clinical trial template(s) 204 include skip logic that dynamically controls the flow of actions, questions, or forms within graphical user interface 210. For example, skip logic evaluates graphical user interface 210 and utilizes conditional statements (e.g., if, else, switch) to determine actions to perform within graphical user interface 210. To illustrate, the clinical trial database generation system 100 can analyze graphical user interface 210 (e.g., using computer-executable instructions 208) by using backend logic to process conditions and retrieve information and determine interactions to perform within graphical user interface 210, such as select element interactions, edit element interactions, or remove element interactions.

In some cases, skip logic of clinical trial template(s) 204 include directions to skip to a specific portion of graphical user interface 210. For example, as previously mentioned, a clinical trial template(s) 204 can correspond to a specific instrument or variable for the clinical trial and skip logic as part of the clinical trial template(s) 204 includes directions to fill in certain portions of graphical user interface (or certain graphical user interfaces) of third-party data collection system.

In one or more embodiments, clinical trial template(s) 204 includes edit checks. In particular, clinical trial template(s) include instructions or predefined rules and/or criteria that validate data. For example, edit checks can include verifying value ranges, required fields, consistency checks, or custom rules for the graphical user interface. Indeed, by using edit checks in clinical trial template(s) 204, the clinical trial database generation system 100 automates generation of clinical trial databases while also ensuring accurate clinical trial databases.

As shown, the clinical trial database generation system 100 utilizes computer-executable instructions to interact with graphical user interface 210 of a third-party data collection system. In particular, computer-executable instructions 208 contain instructions for each graphical user interface of a series of graphical user interfaces of a third-party data collection system. For instance, computer-executable instructions 208 include instructions for identifying one or more elements of graphical user interface 210 and instructions for how to interact with the elements. To illustrate, computer-executable instructions 208 can indicate that graphical user interface 210 comprises a race element, instructions for identifying the race element, and instructions for interacting with the race element based on a clinical trial template and/or a clinical trial standard.

In some embodiments, computer-executable instructions 208 include instructions for interacting with graphical user interfaces of a specific third-party data collection system. In particular, since graphical user interfaces are specific to a third-party data collection system, computer-executable instructions 208 are also specific to a third-party data collection system. Another set of computer-executable instructions will interact with graphical user interfaces of a different third-party data collection system utilizing the clinical template library and clinical trial standards. Indeed, by simply updating the computer-executable instructions, the clinical trial database generation system 100 can flexibly interact with a variety of third-party data collection systems.

As just mentioned, the computer-executable instructions 208 can include instructions for identifying element(s) 212 of graphical user interface 210. Specifically, computer-executable instructions 208 can include instructions for identifying element(s) 212 based on the graphical user interface. For example, for graphical user interface 210 (a specific graphical user interface of the third-party data collection system), computer-executable instructions 208 include instructions for identifying element(s) 212 by identifying specific elements by using visual recognition of the graphical user interface (e.g., with machine-learning model) or by accessing webpage markup of a webpage associated with graphical user interface 210 and identifying webpage markup that corresponds to element(s) 212.

In one or more embodiments, computer-executable instructions 208 include instructions for various interactions with element(s) 212 based on clinical trial template(s) 204 and clinical trial standard 206. Specifically, computer-executable instructions 208 include instructions that identify a clinical trial template (of clinical trial template(s) 204) and/or clinical trial standard 206 that corresponds to an element of element(s) 212 and interactions to perform with the element to update the element to match a clinical trial template or clinical trial standard 206. For example, computer-executable instructions can include instructions perform a remove element interaction 214, edit element interaction 216, or select element interaction 218 based on the clinical trial template or clinical trial standard 206.

For instance, computer-executable instructions 208 include instructions to perform a remove element interaction 214 with element(s) 212. In particular, computer-executable instructions 208 can perform remove element interaction 214 based on identifying that element(s) 212 do not match template data of a clinical trial template or standard data from clinical trial standard 206. In some cases, a remove element interaction 214 includes removing placeholder data of an element that does not match a clinical trial template of clinical trial template(s) 204 or clinical trial standard 206. In other cases, remove element interact removes a portion of element(s) 212 that does not match a clinical trial template of clinical trial template(s) 204 or clinical trial standard 206 (e.g., a race that doesn't match the clinical trial standard 206).

In addition, computer-executable instructions 208 can include instructions to perform an edit element interaction 216. Specifically, computer-executable instructions 208 can perform edit element interaction 216 to edit element(s) 212 based on clinical trial template(s) 204 or clinical trial standard 206. For example, computer-executable instructions can perform an edit element interaction 216 by adding template data from clinical trial template(s) 204 or standard data from clinical trial standard 206.

Further, computer-executable instructions can include instructions to perform a select element interaction 218. Specifically, computer-executable instructions can perform edit element interaction 216 by selecting one or more selectable elements of element(s) 212. For example, computer-executable instructions 208 can make a selection from a drop-down list of element(s) 212 to match clinical trial template(s) 204 or clinical trial standard 206. As another example, computer-executable instructions 208 can select selection indicators of element(s) 212 that match clinical trial template(s) 204 or clinical trial standard 206.

As previously mentioned, the clinical trial database generation system 100 selects clinical trial templates based on configuration data. In some cases, the clinical trial database generation system 100 utilizes machine-learning to extract configuration data and a clinical trial standard version from configuration data. FIG. 3 illustrates a schematic diagram of a clinical trial database generation system utilizing a data-identifying machine-learning model to extract clinical trial specifications and clinical trial standard versions from configuration data in accordance with one or more embodiments.

As shown, the clinical trial database generation system 100 provides configuration data 302 to data-identifying machine-learning model 304 to identify clinical trial specifications 306 and a clinical trial standard 310 within configuration data 302. As used herein, the term “machine-learning model” refers to a computer algorithm or a collection of computer algorithms that automatically improve for a particular task through experience based on the use of data. For example, a machine-learning model can utilize one or more learning techniques to improve accuracy and/or effectiveness. For example, machine-learning models include various types of decision trees, support vector machines, Bayesian networks, or neural networks.

In some embodiments, the data-identifying machine-learning model can be a neural network. The term “neural network” refers to a machine-learning model that can be trained and/or tuned based on inputs to determine classifications or approximate unknown functions. For example, a neural network includes a model of interconnected artificial neurons (e.g., organized in layers) that communicate and learn to approximate complex functions and generate outputs (e.g., generated digital images) based on a plurality of inputs provided to the neural network. In some cases, a neural network refers to an algorithm (or set of algorithms) that implements deep learning techniques to model high-level abstractions in data. For example, a neural network can include a convolutional neural network, a recurrent neural network (e.g., an LSTM), a graph neural network, a self-attention transformer neural network, or a generative adversarial neural network.

As mentioned, the clinical trial database generation system 100 provides configuration data 302 to data-identifying machine-learning model 304. In some cases, configuration data 302 is structured data, such as configuration data generated based on selections within a clinical trial configuration graphical user interface as described below with respect to FIG. 5. In other cases, configuration data 302 is unstructured data and the data-identifying machine-learning model 304 is trained to identify clinical trial specification and a clinical trial standard for a clinical trial from the unstructured data.

As mentioned, the clinical trial database generation system 100 can utilize data-identifying machine-learning model 304 to identify clinical trial specifications 306 from configuration data 302. Specifically, data-identifying machine-learning model 304 is trained or tuned to identify clinical trial specifications 306 from configuration data 302. For example, a data-identifying machine-learning model 304 can identify a type of clinical trial, visit information, instruments to use, and other information for setting up a clinical trial.

Data-identifying machine-learning model 304 can also select clinical trial template(s) 308 based on clinical trial specifications 306. More particularly, data-identifying machine-learning model 304 is trained or tuned to select clinical trial template(s) 308 based on clinical trial specifications 306. For example, if data-identifying machine-learning model 304 identifies that configuration data 302 is for an Alzheimer's drug, data-identifying machine-learning model 304 can select clinical trial template(s) 308, which is used when conducting clinical studies for an Alzheimer's drug. As another example, if data-identifying machine-learning model 304 identifies instruments for collecting clinical data indicated in configuration data 302, data-identifying machine-learning model 304 can select clinical trial template(s) 308 that corresponds to the instruments.

As mentioned, data-identifying machine-learning model 304 identifies a clinical trial standard 310 from configuration data 302. In particular, data-identifying machine-learning model 304 can identify a clinical trial standard version indicated in configuration data 302 and select clinical trial standard 310 from clinical trial standards 312. Clinical trial standards 312 are a plurality of clinical trial standards, where each clinical trial standard is a version of clinical standard, such as various versions of CDISC standards. For example, if the data-identifying machine-learning model 304 identifies that configuration data 302 indicates that a clinical trial will utilize CDISC version 2.0.1, then data-identifying machine-learning model 304 can select CDISC version 2.0.1 for the clinical trial.

As previously mentioned, the clinical trial database generation system 100 interacts with a third-party data collection system to generate a clinical trial database. In addition, the clinical trial database generation system 100 can also generate additional clinical trial requirements for a clinical trial. FIG. 4 illustrates a schematic diagram of a clinical trial database generation system generating clinical trial requirements in accordance with one or more embodiments.

As shown, and as previously described, the clinical trial database generation system 100 utilizes computer-executable instructions 402 that interact with third-party data collection system 406 to generate a clinical trial database using clinical trial template(s) 404 and clinical trial standard 408. In addition to generating a clinical trial database, the clinical trial database generation system 100 generates additional documentation for a clinical trial. In particular, many regulatory agencies require documentation for a clinical trial database indicating the database meets requirements (e.g., FDA requirements or regulations) regarding data integrity, regulatory compliance, and accurate reporting of study results. For example, regulatory (or government) agencies require detailed documentation outlining the structure and content of a clinical trial database, as well as documentation demonstrating the functionality of a clinical trial database, which clinical trial database generation system 100 can also generate.

For example, as shown, the clinical trial database generation system 100 generates clinical trial requirements 410, including clinical trial database 412, specification 414, and validation documentation 416. In particular, the clinical trial database generation system 100 utilizes computer-executable instructions 402 to interact with third-party data collection system 406 and generate clinical trial requirements 410 based on clinical trial template(s) 404 and clinical trial standard 408. For example, as previously described, the clinical trial database generation system 100 generates clinical trial database 412 by using computer-executable instructions 402 to interact with a graphical user interface based on clinical trial template(s) 404 and clinical trial standard 408 to generate clinical trial database 412.

In one or more embodiments, the clinical trial database generation system 100 also utilizes computer-executable instructions 402 to interact with the third-party data collection system to generate specification 414. In some cases, specification 414 is a document required by a government or regulatory agency that indicates detailing the requirements and design of a clinical trial database. The clinical trial database generation system 100 generates specification 414 while also generating clinical trial database 412. For example, the clinical trial database generation system 100 uses computer-executable instructions 402 to access data and information required for specification 414. In some cases, computer-executable instructions 402 access information from clinical trial template(s) 404 and clinical trial standard 408 to generate specification 414. In some instances, the clinical trial database generation system 100 generates specification 414 while generating clinical trial database 412. In other instances, the clinical trial database generation system 100 generates specification 414 after generating clinical trial database 412.

In addition to specification 414, the clinical trial database generation system 100 also generates validation documentation 416. In some cases, validation documentation 416 is documentation required by a government or regulatory agency demonstrating the functionality of a clinical trial database. The clinical trial database generation system 100 can utilize computer-executable instructions 402 to generate validation documentation 416. For example, the clinical trial database generation system 100 uses computer-executable instructions 402 to generate screenshots of clinical trial database 412 by using computer-executable instructions 402 to take (or instruct another system to take) screenshots of graphical user interfaces. To illustrate, computer-executable instructions 402 can include instructions to interact with a graphical user interface to include template data from clinical trial template(s) 404 and/or standard data from clinical trial standard 408 and, after the interactions are complete, take a screenshot of the graphical user interface. The clinical trial database generation system 100 can utilize the screenshots to demonstrate that clinical trial database 412 works and that it uses the same information indicated in specification 414. Indeed, because the clinical trial database generation system 100 generates clinical trial database 412, specification 414, and validation documentation 416 using computer-executable instructions 402 to access the same information (e.g., the clinical trial template(s) and the clinical trial standard 408), the clinical trial database generation system 100 increases accuracy over other systems that pull documentation from various systems and are prone to mistakes and missing information.

As previously mentioned, the clinical trial database generation system 100 receives configuration data. In some cases, the clinical trial database generation system 100 receives configuration data based on selections within a configuration graphical user interface. FIG. 5 illustrates an example graphical user interface of a configuration graphical user interface of the clinical trial database generation system 100 for receiving configuration data in accordance with one or more embodiments.

As shown in FIG. 5, the clinical trial database generation system 100 includes a configuration graphical user interface 500. In particular, the clinical trial database generation system 100 receives configuration data by receiving selections within configuration graphical user interface 500 from an administrator device of a clinical trial. For example, the administrator device selects options within configuration graphical user interface corresponding to various aspects of a clinical trial, including, but not limited to, visits, instruments used at each visit (e.g., questionnaires, tests, or other means of collecting data), or types of data collected at each visit. In some cases, configuration graphical user interface is included within the clinical trial database generation system 100. In other cases, configuration graphical user interface is a graphical user interface of a third-party system used to gather selections of options (e.g., a Google Doc, Google Spreadsheet, or Google Form).

As illustrated, configuration graphical user interface 500 includes visit list 502. Specifically, visit list 502 includes various clinical visits of the clinical trial and an administrator device can select (or input) various visits at which the administrators of the clinical trial will collect clinical data. For example, the configuration graphical user interface can include options for selecting pre-defined visits of the clinical trial. As another example, the configuration graphical user interface can include options for an administrator device to input visits with names that are unique to the clinical trial.

As also shown, the configuration graphical user interface 500 includes an option 504 to select a clinical trial standard for the clinical study. Specifically, configuration graphical user interface 500 includes a predefined list of clinical trial standards and the administrator device selects the clinical trial standard for the clinical trial. For example, option 504 can include a listing of standards from the Clinical Data Interchange Standards Consortium (CDISC) and an administrator device selects the date (or version) of the CDISC standard for the clinical trial.

As also illustrated, configuration graphical user interface 500 includes options 506 for receiving selections of data for each visit. In particular, options 506 include a listing of visits indicated in visit list 502 and options to select data collected at the visit, instruments to use during the visit, or testing to perform during the visit. For example, as shown, based on selections in options 506, during a screening visit (e.g., a visit to establish whether a patient qualifies for a clinical trial), clinical trial administrator will collect a visit date, demographics, inclusion/exclusion criteria, medical history and vital signs and perform a physical examination. However, during a baseline visit a clinical trial administrator will collect a visit date and vital signs and conduct a physical examination and during a week one visit will only collect a visit date and vital signs.

In one or more embodiments, options 506 are associated with clinical trial templates and the clinical trial database generation system 100 selects one or more clinical trial templates based on selections of options 506. For example, options 506 can include selectable options for different instruments for collecting data during the clinical trial and selecting the option selects a clinical trial template that corresponds to the selected instrument. To illustrate, if a clinical trial is going to use an MMSE screening for an initial visit or baseline visit, an administrator device can select a corresponding option for the MMSE screening, which selects a clinical trial template corresponding to the MMSE screening for computer-executable instructions to use when generating the clinical trial database for the initial or baseline visits. If a clinical trial is going to use an ADAS-Cog screening for subsequent visits (e.g., after initial or baseline visits), an administrator device can select an option for the ADAS-Cog screening, which selects a clinical trial template corresponding to the ADAS-Cog screening for computer-executable instructions to use when generating the clinical trial database for the subsequent visits.

As previously mentioned, the clinical trial database generation system 100 performs select element interaction with a graphical user interface of a third-party data collection system. In particular, the clinical trial database generation system 100 can utilize computer-executable instructions to interact with elements of a graphical user interface. FIG. 6 illustrates an example graphical user interface of a clinical trial database generation system utilizing computer-executable instructions to interact with elements of a graphical user interface based on corresponding clinical trial template(s) or a clinical trial standard in accordance with one or more embodiments.

In one or more embodiments, as indicated in FIG. 6, clinical trial database generation system 100 utilizes computer-executable instructions to identify element 602 of graphical user interface 600 of a third-party data collection system. In particular, computer-executable instructions identify element 602 by identifying a specific element indicated in the instructions. For example, computer-executable instructions can identify an element by identifying webpage markup (e.g., HTML) of a webpage associated with graphical user interface 600.

As further shown, after identifying element 602, computer-executable instructions interact with element 602. Specifically, computer-executable instructions include instructions for interacting with element 602, including an edit element interaction, a select element interaction, or a remove element interaction, as discussed with respect to FIG. 2 above. As illustrated, computer-executable instructions select a template from a drop-down menu of element 602.

As indicated in FIG. 6, the clinical trial database generation system 100 can use computer-executable instructions to select various outside documents associated with element 602. Specifically, computer-executable instructions can include instructions for which form, clinical trial template, or other document to select for element 602. In some cases, the computer-executable instructions include instructions to use a specific form, clinical trial template, or other document for element 602. In other cases, computer-executable instructions include instructions to select a form, clinical trial template, or other document based on indications in configuration data.

As previously mentioned, the clinical trial database generation system 100 performs a select element interaction with a graphical user interface of a third-party data collection system. In particular, the clinical trial database generation system 100 utilizes computer-executable instructions to make selections of options within an element of a graphical user interface of a third-party data collection system. FIG. 7 illustrates an example graphical user interface of the clinical trial database generation system 100 utilizing computer-executable instructions to make selections within an element in accordance with one or more embodiments.

As illustrated in FIG. 7, the clinical trial database generation system 100 interacts with graphical user interface 700 of the third-party data collection system. In particular, the clinical trial database generation system 100 utilizes computer-executable instructions to interact with graphical user interface and identify element 702 that includes options to make selections within element 702. For example, as shown, element 702 can include options that, when selected, generate portions of a clinical trial database. To illustrate, as shown, element 702 can include options to select visits corresponding to clinical visits (e.g., where a patient of the clinical trial visits a clinical trial administrator or doctor).

In one or more embodiments, the clinical trial database generation system 100 selects options in element 702 based on clinical trial template(s) and/or a clinical trial standard. Specifically, the clinical trial database generation system 100 utilizes computer-executable instructions that access clinical trial template(s) and/or a clinical trial standard to determine what options of element 702 to select. In some cases, the clinical template(s) include information from configuration data that is specific to the clinical trial (e.g., visits for the clinical trial).

As previously mentioned, the clinical trial database generation system 100 can perform a remove element interaction and/or an edit element interaction with an element of a graphical user interface of a third-party data collection system. In particular, the clinical trial database generation system 100 utilizes computer-executable instructions to perform a remove element interaction and/or an edit element interaction with an element of a graphical user interface of a third-party data collection system based on clinical trial template(s) and/or a clinical trial standard. FIGS. 8A-8C illustrates an example graphical user interface of the clinical trial database generation system 100 interacting with and modifying an element in accordance with one or more embodiments. Specifically, FIG. 8A illustrates the clinical trial database generation system 100 identifying elements of a graphical user interface and comparing placeholder data of the elements to clinical trial templates, FIG. 8B illustrates the clinical trial database generation system 100 performing a remove element interaction by removing elements of the graphical user interface, and FIG. 8C illustrates the clinical trial database generation system 100 performing an edit element interaction by adding elements to the graphical user interface.

As shown in FIG. 8A, the clinical trial database generation system 100 utilizes computer-executable instructions to identify elements 802 in graphical user interface 800. In particular, the clinical trial database generation system 100 uses computer-executable instructions to identify elements 802 and compare placeholder data (or content and/or data) of elements 802 to clinical trial template(s) and/or a clinical trial standard. For instance, computer-executable instructions can include instructions that indicate a specific clinical trial template to compare the placeholder data. In other instances, the computer-executable instructions can include instructions to compare the placeholder data to a clinical trial standard for the clinical trial (e.g., the clinical trial standard indicated in configuration data).

The clinical trial database generation system 100 can determine an action for elements 802 based on comparing placeholder elements to a clinical trial template and/or a clinical trial standard and computer-executable instructions. Specifically, computer-executable instructions for the graphical user interface include instructions for how to interact with elements 802 if the placeholder elements do not match the clinical trial template or the clinical trial standard. For example, the clinical trial database generation system 100 might determine that a category does not match the clinical trial template based on identifying that the clinical trial template and/or clinical trial standard. The clinical trial database generation system 100 then utilizes the computer executable instructions to instruct the third-party data collection system to add a category to elements 802.

The clinical trial database generation system 100 also includes instructions for removing the element based on the computer-executable instructions. Specifically, computer-executable instructions for graphical user interface include instructions for how to interact with elements 802 if the placeholder elements do not match the clinical trial template or clinical trial standard. Because computer-executable instructions are unique to graphical user interface, the instructions for how to interact with elements 802 will correspond to a type of element 802. For example, if elements 802 are not editable, computer-executable instructions can include instructions for the third-party data collection system remove elements 802. As another example, computer-executable instructions can include instructions for the third-party data collection system to remove text of elements 802 and replace text that matches the clinical trial template or the clinical trial standard.

As shown in FIG. 8B, the clinical trial database generation system 100 utilizes computer-executable instructions to perform a remove element interaction and removes elements 802 from graphical user interface 800 by removing elements 802. Specifically, the clinical trial database generation system 100 identifies that placeholder elements (and placeholder data or placeholder content) do not match the clinical data template or the clinical trial standard and removes elements 802 from the graphical user interface. For example, computer-executable instructions can perform a remove element action to remove elements 802 by selecting an option to remove elements within graphical user interface 800. Conversely, in some embodiments, if elements 802 are editable, the clinical trial database generation system 100 can edit elements 802 to match the clinical trial template and/or the clinical trial standard rather than removing elements 802.

As also shown in FIG. 8B, the clinical trial database generation system 100 identifies element 804 to add an element to graphical user interface 800. In particular, the clinical trial database generation system 100 uses computer-executable instructions to identify element 804. For example, computer-executable instructions include instructions to identify element 804 in order to add template data from the clinical trial template or standard data from the clinical trial standard to graphical user interface 800 (e.g., to generate the clinical trial database).

As shown in FIG. 8C, the clinical trial database generation system 100 adds elements 806 to match the clinical trial template and/or the clinical trial standard. Specifically, the clinical trial database generation system 100 uses computer-executable instructions to select element 804 from FIG. 8B and add elements 806 that include information that matches the clinical trial template and/or the clinical trial standard. For example, the clinical trial database generation system 100 can add elements that include different information, such as changing the category or a value of the elements. As another example, the clinical trial database generation system 100 adds options that were not included in elements 804, such as adding a category.

In some embodiments, the clinical trial database generation system 100 compares elements to a clinical trial template and the clinical trial template indicates that the data is from a clinical trial standard. Specifically, computer-executable instructions access the clinical trial template, and the clinical trial template accesses the information from a clinical trial standard indicated in configuration data. For example, for demographic information (e.g., race, ethnicity, sex), a clinical trial template will indicate that the data is found in a clinical trial standard, and the computer-executable instructions access the information in the version of clinical trial standard indicated in the configuration data.

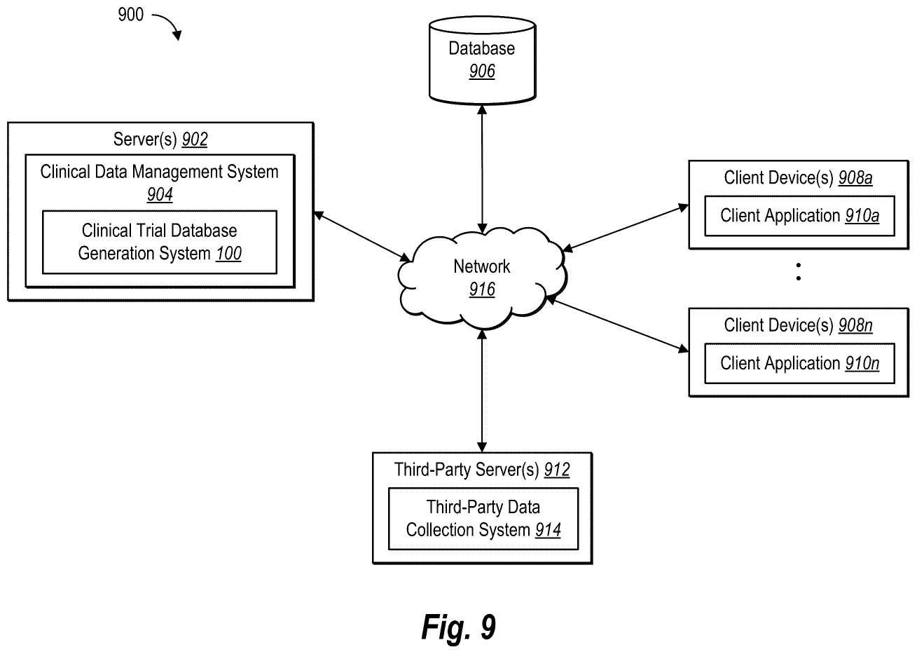

As previously mentioned, the clinical trial database generation system 100 generates a clinical trial database by using computer-executable instructions to interact with a graphical user interface of a third-party data collection system to generate a clinical database. In particular, the clinical trial database generation system utilizes various devices, servers, and networks for storing, synchronizing, and communicating regarding generating clinical databases. FIG. 9 illustrates a schematic diagram of an environment in which a clinical trial database generation system 100 can operate in accordance with one or more embodiments.

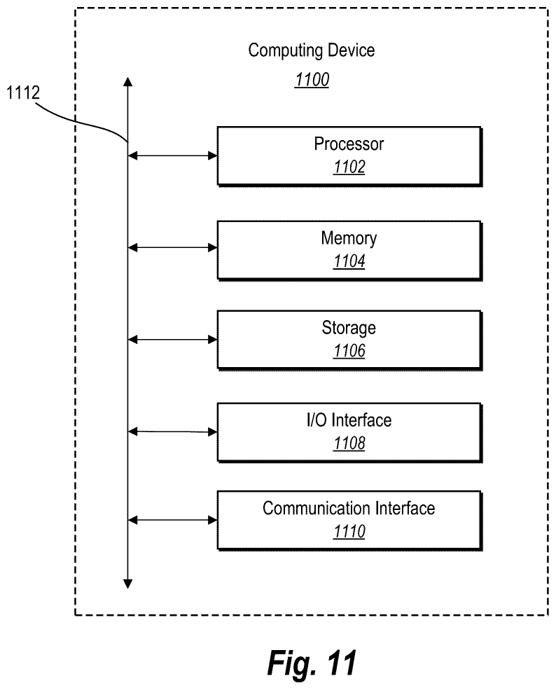

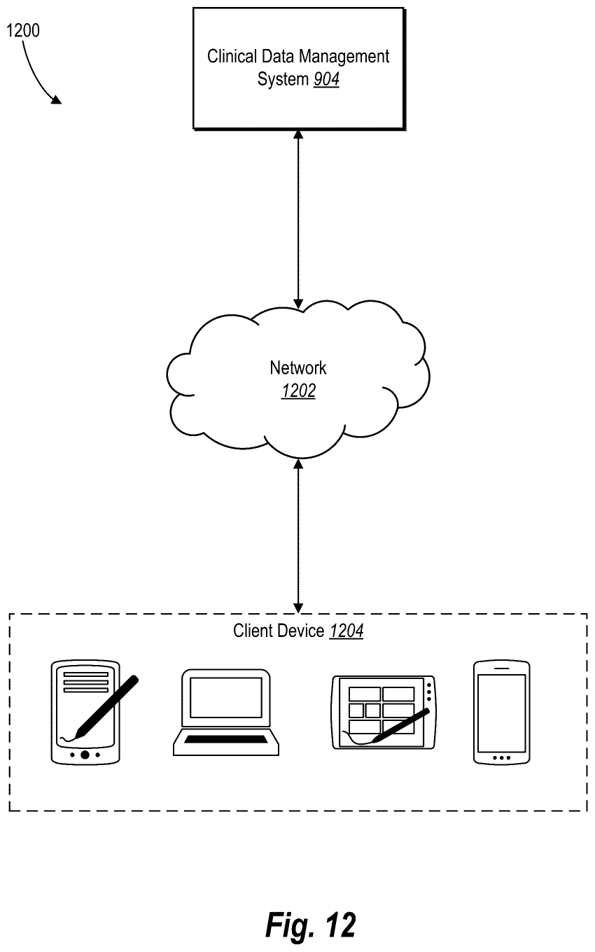

As shown, the environment 900 includes server(s) 902, database 906, client device(s) 908a-908n, and third-party server(s) 912. Each of the components of the environment 900 can communicate via network 916, and network 916 may be any suitable network over which computing devices can communicate. Example networks are discussed in more detail in relation to FIGS. 11-12.

As mentioned above, the environment 900 includes client device(s) 908a-908n. The client device(s) 908a-908n can be one of a variety of computing devices, including a smartphone, a tablet, a smart television, a desktop computer, a laptop computer, a virtual reality device, an augmented reality device, or another computing device as described in relation to FIGS. 11-12. The client device(s) 908a-908n can communicate with the server(s) 902 via network 916. For example, the client device(s) 908a-908n can receive user input from a user interacting with client device(s) 908a-908n (e.g., via the client application(s) 910a-910n) to, for instance, receive user interactions with content items. In addition, the clinical trial database generation system 100 or the server(s) 902 can receive information relating to various interactions with content items and/or user interface elements based on the input received by the client device(s) 908a-908n.

As shown, client device(s) 908a-908n can include client application(s) 910a-910n. In particular, client application(s) 910a-910n may be a web application, a native application installed on the client device(s) 908a-908n (e.g., a mobile application, a desktop application, etc.), or a cloud-based application where all or part of the functionality is performed by the server(s) 902. Based on instructions from client application(s) 910a-910n, the client device(s) 908a-908n can present or display information, including a user interface for interacting with a third-party data collection system 914. Using client application(s) 910a-910n, the client device(s) 908a-908n can perform (or request to perform) various operations, such as displaying information relating to clinical databases, clinical trial templates, clinical trial standards and/or the third-party data collection system 914.

As illustrated in FIG. 9, the environment 900 also includes the server(s) 902. The server(s) 902 may generate, track, store, process, receive, and transmit electronic data, such as clinical trials (and related template data), clinical trial standard (and related standard data), interactions with interface elements, and/or interactions between user accounts or client devices. For example, the server(s) 902 may receive an indication from the client device(s) 908a-908n of a user interaction, such as entering configuration data for a clinical trial. In addition, the server(s) 902 can transmit data to the client device(s) 908a-908n in the form of a graphical user interface or other depiction. Indeed, the server(s) 902 can communicate with the client device(s) 908a-908n to send and/or receive data via network 916. In some implementations, the server(s) 902 comprise(s) a distributed server where the server(s) 902 include(s) a number of server devices distributed across the network 916 and located in different physical locations. The server(s) 902 can comprise one or more content servers, application servers, container orchestration servers, communication servers, web-hosting servers, machine-learning servers, and other types of servers.

As shown in FIG. 9, the server(s) 902 can also include clinical trial database generation system 100 as part of clinical data management system 904. The clinical data management system 904 can communicate with the client device(s) 908a-908n to perform various functions associated with the client application(s) 910a-910n, such as managing user accounts, receiving configuration data, or receiving other data related to a clinical trial. In some embodiments, the clinical trial database generation system and/or the clinical data management system 904 utilize the database 906 to store and access information such as clinical trial templates or clinical trial standards.

In addition, as shown in FIG. 9, the environment 900 can also include third-party server(s) 912 hosting third-party data collection system 914. In particular, the third-party data collection system 914 that enables the efficient gathering and storage of data by providing a graphical user interface that provides real-time access to organized datasets. For example, the clinical trial database generation system 100 can interact with the third-party data collection system 914 to generate a clinical trial database that includes variables and other information for a clinical trial.

Although FIG. 9 depicts the clinical trial database generation system located on the server(s) 902, in some implementations, the clinical trial database generation system may be implemented by (e.g., located entirely or in part on) one or more other components of the environment. For example, the clinical trial database generation system may be implemented as part of client device(s) 908a-908n and/or a third-party system. As another example, the client device(s) 908a-908n and/or a third-party system can download all or part of the clinical trial database generation system for implementation independent of, or together with, the server(s) 902.

In some implementations, though not illustrated in FIG. 9, the environment 900 may have a different arrangement of components and/or may have a different number or set of components altogether. For example, the client device(s) 908a-908n may communicate directly with the clinical trial database generation system, bypassing network 916. The environment 900 may also include one or more third-party systems, each corresponding to a different data source. In addition, the environment 900 can include the database 906 located external to the server(s) 902 (e.g., in communication via the network 916) or located on the server(s) 902 and/or on the client device(s) 908a-908n.

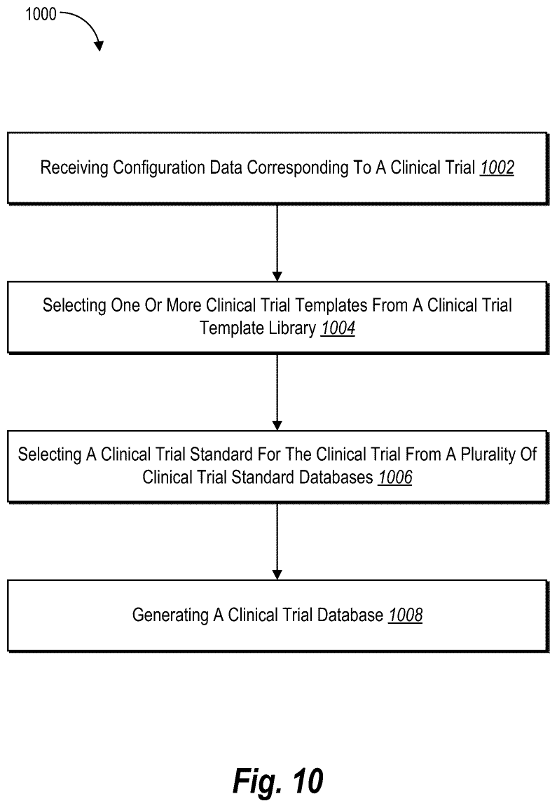

FIGS. 1-9, the corresponding text, and the examples provide a number of different methods, systems, devices, and non-transitory computer-readable media of the clinical trial database generation system. In addition to the foregoing, one or more embodiments can also be described in terms of flowcharts comprising acts for accomplishing a particular result, as shown in FIG. 10. FIG. 10 may be performed with more or fewer acts. Further, the acts may be performed in differing orders. Additionally, the acts described herein may be repeated or performed in parallel with one another or parallel with different instances of the same or similar acts.

As mentioned, FIG. 10 illustrates a flowchart of a series of acts 1000 for generating a clinical trial database by utilizing computer-executable instructions to interact with a third-party data collection system using clinical trial templates and clinical trial standards in accordance with one or more embodiments. While FIG. 10 illustrates acts according to one embodiment, alternative embodiments may omit, add to, reorder, and/or modify any of the acts shown in FIG. 10. The acts of FIG. 10 can be performed as part of a method. Alternatively, a non-transitory computer-readable medium can comprise instructions that, when executed by one or more processors, cause a computing device to perform the acts of FIG. 10. In some embodiments, a system can perform the acts of FIG. 10.

As shown in FIG. 10, the series of acts 1000 includes an act 1002 of receiving configuration data corresponding to a clinical trial, an act 1004 of selecting one or more clinical trial templates from a clinical trial template library, an act 1006 of selecting a clinical trial standard database for the clinical trial from a plurality of clinical trial standards, and an act 1008 of generating a clinical trial database.

In particular, the act 1002 can include receiving configuration data corresponding to a clinical trial, the act 1004 can include selecting, based on the configuration data, one or more clinical trial templates from a clinical trial template library, the act 1006 can include selecting, based on the configuration data, a clinical trial standard for the clinical trial from a plurality of clinical trial standards, and the act 1008 can include generating, based on the one or more clinical trial templates and the clinical trial standard, a clinical trial database for the clinical trial by utilizing computer-executable instructions to interact with a third-party data collection system.

In one or more embodiments, the series of acts 1000 includes identifying an element of a graphical user interface of the third-party data collection system and adding template data from the one or more clinical trial templates or standard data from the clinical trial standard to the element. Further, in one or more embodiments, the series of acts 1000 includes identifying webpage markup of a webpage corresponding to the graphical user interface that identifies the element.

Further, in some embodiments, the series of acts 1000 includes identifying an element of a graphical user interface of the third-party data collection system, identifying that a clinical data template of the clinical trial template library corresponds to the element, and based on identifying that the clinical data template corresponds to the element, adding template data from the clinical data template to the element. Moreover, in one or more embodiments, the series of acts 1000 includes identifying that the clinical data template of the clinical trial template library corresponds to the element further comprises: identifying that the element corresponds to a specific clinical data template of the clinical trial template library; and determining that the clinical data template is the specific clinical data template.

Moreover, in one or more embodiments, the series of acts 1000 includes identifying an element of the third-party data collection system that corresponds to a clinical trial standard; determining that the element does not match the clinical trial standard for the clinical trial; and updating the element to match the clinical trial standard for the clinical trial. In addition, in one or more embodiments, the series of acts 1000 includes removing placeholder data of the element and adding standard data corresponding to the clinical trial standard to the element.

Also, in some embodiments, the series of acts 1000 includes identifying a clinical standard version indicated in the configuration data, and selecting the clinical trial standard for the clinical trial based on identifying that the clinical trial standard is the clinical standard version indicated in the configuration data.

Further, in one or more embodiments, the series of acts 1000 include wherein the computer-executable comprise instructions for identifying and interacting with elements of graphical user interfaces of the third-party data collection system, instructions identifying template data of the one or more clinical trial templates or standard data of the clinical trial standard corresponding to the elements of the graphical user interfaces, and instructions for interacting with the elements of the graphical user interfaces utilizing the template data or the standard data.

In addition, in some embodiments, the series of acts include identifying an element of a graphical user interface of the third-party data collection system and adding template data from the one or more clinical trial templates or standard data from the clinical trial standard to the element. Moreover, in one or more embodiments, the series of acts 1000 includes identifying an element of the third-party data collection system that corresponds to a clinical trial standard and determining that the element does not match the clinical trial standard for the clinical trial and updating the element to match the clinical trial standard for the clinical trial.

Moreover, in one or more embodiments, the series of acts 1000 includes identifying an element of a graphical user interface of the third-party data collection system, identifying that the element corresponds to a specific clinical trial template of the clinical trial template library, and based on identifying that the element corresponds to the specific clinical trial template, adding data from the specific clinical trial template to the element.

Also, in some embodiments, the series of acts 1000 includes identifying an element of a graphical user interface of the third-party data collection system and updating the element by removing a placeholder data of the element and adding template data or standard data to the element.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 11 illustrates a block diagram of an example computing device 1100 that may be configured to perform one or more of the processes described above. One will appreciate that one or more computing devices, such as the computing device 1100 may represent the computing devices described above (e.g., client device(s) 908a-908n, server(s) 902, and third-party server(s) 912). In one or more embodiments, the computing device 1100 may be a mobile device (e.g., a mobile telephone, a smartphone, a PDA, a tablet, a laptop, a camera, a tracker, a watch, a wearable device, etc.). In some embodiments, the computing device 1100 may be a non-mobile device (e.g., a desktop computer or another type of client device). Further, the computing device 1100 may be a server device that includes cloud-based processing and storage capabilities.

As shown in FIG. 11, the computing device 1100 can include one or more processor(s) 1102, memory 1104, a storage device 1106, input/output interfaces 1108 (or “I/O interfaces 1108”), and a communication interface 1110, which may be communicatively coupled by way of a communication infrastructure (e.g., bus 1112). While the computing device 1100 is shown in FIG. 11, the components illustrated in FIG. 11 are not intended to be limiting. Additional or alternative components may be used in other embodiments. Furthermore, in certain embodiments, the computing device 1100 includes fewer components than those shown in FIG. 11. Components of the computing device 1100 shown in FIG. 11 will now be described in additional detail.

In particular embodiments, the processor(s) 1102 includes hardware for executing instructions, such as those making up a computer program. As an example, and not by way of limitation, to execute instructions, the processor(s) 1102 may retrieve (or fetch) the instructions from an internal register, an internal cache, memory 1104, or a storage device 1106 and decode and execute them.

The computing device 1100 includes memory 1104, which is coupled to the processor(s) 1102. The memory 1104 may be used for storing data, metadata, and programs for execution by the processor(s). The memory 1104 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1104 may be internal or distributed memory.

The computing device 1100 includes a storage device 1106 includes storage for storing data or instructions. As an example, and not by way of limitation, the storage device 1106 can include a non-transitory storage medium described above. The storage device 1106 may include a hard disk drive (HDD), flash memory, a Universal Serial Bus (USB) drive or a combination these or other storage devices.

As shown, the computing device 1100 includes one or more I/O interfaces 1108, which are provided to allow a user to provide input to (such as user strokes), receive output from, and otherwise transfer data to and from the computing device 1100. These I/O interfaces 1108 may include a mouse, keypad or a keyboard, a touch screen, camera, optical scanner, network interface, modem, other known I/O devices or a combination of such I/O interfaces 1108. The touch screen may be activated with a stylus or a finger.

The I/O interfaces 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1108 are configured to provide graphical data to a display for presentation to a user. The graphical data may be representative of one or more graphical user interfaces and/or any other graphical content as may serve a particular implementation.

The computing device 1100 can further include a communication interface 1110. The communication interface 1110 can include hardware, software, or both. The communication interface 1110 provides one or more interfaces for communication (such as, for example, packet-based communication) between the computing device and one or more other computing devices or one or more networks. As an example, and not by way of limitation, communication interface 1110 may include a network interface controller (NIC) or network adapter for communicating with an Ethernet or other wire-based network or a wireless NIC (WNIC) or wireless adapter for communicating with a wireless network, such as a WI-FI. The computing device 1100 can further include a bus 1112. The bus 1112 can include hardware, software, or both that connects components of computing device 1100 to each other.

FIG. 12 illustrates an example network environment 1200 of a clinical data management system 904 (e.g., the clinical data management system 904, including the clinical trial database generation system 100). The network environment 1200 includes a clinical data management system 904 and a client device 1204, connected to each other by a network 1202. Although FIG. 12 illustrates a particular arrangement of the client device 1204, the clinical data management system 904, and the network 1202, this disclosure contemplates any suitable arrangement of the client device 1204, the clinical data management system 904, and the network 1202. As an example, and not by way of limitation, two or more of the client devices 1204 and the clinical data management system 904 communicate directly, bypassing the network 1202. As another example, two or more of the client devices 1204 and the clinical data management system 904 may be physically or logically co-located with each other in whole or in part. Moreover, although FIG. 12 illustrates a particular number of the client device 1204, the clinical data management system 904, and the network 1202, this disclosure contemplates any suitable number of client devices 1204, clinical data management systems 904, and networks 1202. As an example, and not by way of limitation, the network environment 1200 may include multiple client devices 1204, multiple clinical management systems 904, and multiple networks 1202.

This disclosure contemplates any suitable network 1202. As an example, and not by way of limitation, one or more portions of the network 1202 may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these. The network 1202 may include one or more networks 1202.

Links may connect the client device 1204 and the clinical data management system 904 to the network 1202 or to each other. This disclosure contemplates any suitable links. In particular embodiments, one or more links include one or more wireline (such as, for example, Digital Subscriber Line (“DSL”) or Data Over Cable Service Interface Specification (“DOCSIS”)), wireless (such as, for example, Wi-Fi or Worldwide Interoperability for Microwave Access (“WiMAX”)), or optical (such as, for example, Synchronous Optical Network (“SONET”) or Synchronous Digital Hierarchy (“SDH”)) links. In particular embodiments, one or more links each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link, or a combination of two or more such links. Links need not necessarily be the same throughout the network environment 1200. One or more first links may differ in one or more respects from one or more second links.

In particular embodiments, the client device 1204 may be an electronic device including hardware, software, or embedded logic components or a combination of two or more such components and capable of carrying out the appropriate functionalities implemented or supported by the client device 1204. As an example, and not by way of limitation, a client device 1204 may include any of the computing devices discussed above in relation to FIG. 9. A client device 1204 may enable a network user at the client device 1204 to access a network. A client device 1204 may enable its user to communicate with other users at other client devices 1204. A client device 1204 can be the client device(s) 908a-908n.

In particular embodiments, the client device 1204 may include a web browser, such as GOOGLE CHROME or MOZILLA FIREFOX, and may have one or more add-ons, plug-ins, or other extensions. A user at the client device 1204 may enter a Uniform Resource Locator (“URL”) or other address directing the web browser to a particular server (such as the server(s) 902), and the web browser may generate a Hyper Text Transfer Protocol (“HTTP”) request and communicate the HTTP request to the server. The server may accept the HTTP request and communicate to the client device 1204 one or more Hyper Text Markup Language (“HTML”) files responsive to the HTTP request. The client device 1204 may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

The clinical data management system 904 may be accessed by the other components of the network environment 1200 either directly or via network 1202. In particular embodiments, the clinical data management system 904 may include one or more servers. Each server may be a unitary server or a distributed server spanning multiple computers or multiple datacenters. Servers may be of various types, such as, for example and without limitation, web server, news server, mail server, message server, advertising server, file server, application server, exchange server, database server, proxy server, another server suitable for performing functions or processes described herein, or any combination thereof. In particular embodiments, each server may include hardware, software, or embedded logic components or a combination of two or more such components for carrying out the appropriate functionalities implemented or supported by server. In particular embodiments, the clinical data management system 904 may include one or more data stores. Data stores may be used to store various types of information. In particular embodiments, the information stored in data stores may be organized according to specific data structures. In particular embodiments, each data store may be a relational, columnar, correlation, or other suitable database. Although this disclosure describes or illustrates particular types of databases, this disclosure contemplates any suitable types of databases. Particular embodiments may provide interfaces that enable the client device 1204 or the clinical data management system 904 to manage, retrieve, modify, add, or delete, the information stored in data storage.

In particular embodiments, the clinical data management system 904 may be capable of linking a variety of entities. As an example, and not by way of limitation, the clinical data management system 904 may enable multiple users and/or agents to interact with each other or other entities, or to allow users and/or agents to interact with these entities through an application programming interface (“API”) or other communication channels.

In particular embodiments, the clinical data management system 904 may include a variety of servers, sub-systems, programs, modules, logs, and data stores. In particular embodiments, the clinical data management system 904 may include one or more of the following: a web server, action logger, API-request server, relevance-and-ranking engine, content-object classifier, notification controller, action log, third-party-content-object-exposure log, inference module, authorization/privacy server, search module, advertisement-targeting module, user-interface module, user-profile store, connection store, third-party content store, or location store. The clinical data management system 904 may also include suitable components such as network interfaces, security mechanisms, load balancers, failover servers, management-and-network-operations consoles, other suitable components, or any suitable combination thereof.

In particular embodiments, the clinical data management system 904 may include one or more user-profile stores for storing user profiles. A user profile may include, for example, biographic information, demographic information, behavioral information, social information, or other types of descriptive information, such as work experience, educational history, hobbies or preferences, interests, affinities, or location. Interest information may include interests related to one or more categories. Categories may be general or specific. Additionally, a user profile may include financial and billing information of users (e.g., customers, etc.).

The web server may include a mail server or other messaging functionality for receiving and routing messages between the clinical data management system 904 and one or more client devices 1204. An action logger may be used to receive communications from a web server about a user's actions on or off the clinical data management system 904. In conjunction with the action log, a third-party-content-object log may be maintained of user exposures to third-party-content objects. A notification controller may provide information regarding content objects to the client device 1204. Information may be pushed to the client device 1204 as notifications, or information may be pulled from the client device 1204 responsive to a request received from the client device 1204. Authorization servers may be used to enforce one or more privacy settings of the users of the clinical data management system 904. A privacy setting of a user determines how particular information associated with a user can be shared. The authorization server may allow users to opt in to or opt out of having their actions logged by the clinical data management system 904 or shared with other systems, such as, for example, by setting appropriate privacy settings. Third party-content-object stores may be used to store content objects received from third parties. Location stores may be used for storing location information received from the client devices 1204 associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
receiving configuration data corresponding to a clinical trial;
selecting, based on the configuration data, one or more clinical trial templates from a clinical trial template library;
selecting, based on the configuration data, a clinical trial standard for the clinical trial from a plurality of clinical trial standards; and
generating, based on the one or more clinical trial templates and the clinical trial standard, a clinical trial database for the clinical trial by utilizing computer-executable instructions to interact with a third-party data collection system.

2. The computer-implemented method of claim 1, wherein utilizing the computer-executable instructions to interact with the third-party data collection system further comprises:
identifying an element of a graphical user interface of the third-party data collection system; and
adding template data from the one or more clinical trial templates or standard data from the clinical trial standard to the element.

3. The computer-implemented method of claim 2, wherein identifying the element of the graphical user interface of the third-party data collection system further comprises identifying webpage markup of a webpage corresponding to the graphical user interface that identifies the element.

4. The computer-implemented method of claim 1, further comprising:
identifying an element of a graphical user interface of the third-party data collection system;
identifying that a clinical data template of the clinical trial template library corresponds to the element; and
based on identifying that the clinical data template corresponds to the element, adding template data from the clinical data template to the element.

5. The computer-implemented method of claim 4, wherein identifying that the clinical data template of the clinical trial template library corresponds to the element further comprises:
identifying that the element corresponds to a specific clinical data template of the clinical trial template library; and
determining that the clinical data template is the specific clinical data template.

6. The computer-implemented method of claim 1, wherein utilizing the computer-executable instructions to interact with the third-party data collection system further comprises:
identifying an element of the third-party data collection system that corresponds to a clinical trial standard;
determining that the element does not match the clinical trial standard for the clinical trial; and
updating the element to match the clinical trial standard for the clinical trial.

7. The computer-implemented method of claim 6, wherein updating the element to match to the clinical trial standard for the clinical trial further comprises:
removing placeholder data of the element; and
adding standard data corresponding to the clinical trial standard to the element.

8. The computer-implemented method of claim 1, wherein selecting the clinical trial standard for the clinical trial from the plurality of clinical trial standards further comprises:
identifying a clinical standard version indicated in the configuration data; and
selecting the clinical trial standard for the clinical trial based on identifying that the clinical trial standard is the clinical standard version indicated in the configuration data.

9. The computer-implemented method of claim 1, wherein the computer-executable instructions comprise:
instructions for identifying and interacting with elements of graphical user interfaces of the third-party data collection system;
instructions identifying template data of the one or more clinical trial templates or standard data of the clinical trial standard corresponding to the elements of the graphical user interfaces; and
instructions for interacting with the elements of the graphical user interfaces utilizing the template data or the standard data.

10. A non-transitory computer-readable medium storing instructions that, when executed by at least one processor, cause a computer system to:
receive configuration data corresponding to a clinical trial;
select, based on the configuration data, one or more clinical trial templates from a clinical trial template library;
select, based on the configuration data, a clinical trial standard for the clinical trial from a plurality of clinical trial standards; and
generate, based on the one or more clinical trial templates and the clinical trial standard, a clinical trial database for the clinical trial by utilizing computer-executable instructions to interact with a third-party data collection system.

11. The non-transitory computer-readable medium of claim 10, further comprising instructions that, when executed by the at least one processor, cause the computer system to utilize the computer-executable instructions to interact with the third-party data collection system by:
identifying an element of a graphical user interface of the third-party data collection system; and
adding template data from the one or more clinical trial templates or standard data from the clinical trial standard to the element.

12. The non-transitory computer-readable medium of claim 11, wherein identifying the element of the graphical user interface of the third-party data collection system further comprises identifying that webpage markup of a webpage corresponding to the graphical user interface corresponds to the element.

13. The non-transitory computer-readable medium of claim 10, further comprising instructions that, when executed by the at least one processor, cause the computer system to:
identify an element of a graphical user interface of the third-party data collection system;
identify that a clinical data template of the clinical trial template library corresponds to the element; and
based on identifying that the clinical data template corresponds to the element, add data from the clinical data template to the element.

14. The non-transitory computer-readable medium of claim 10, further comprising instructions that, when executed by the at least one processor, cause the computer system to utilize the computer-executable instructions to interact with the third-party data collection system by:
identifying an element of the third-party data collection system that corresponds to a clinical trial standard;
determining that the element does not match the clinical trial standard for the clinical trial; and
updating the element to match the clinical trial standard for the clinical trial.

15. The non-transitory computer-readable medium of claim 10, further comprising instructions that, when executed by the at least one processor, cause the computer system to select the clinical trial standard for the clinical trial from the plurality of clinical trial standards by:
identifying a clinical standard version indicated in the configuration data; and
selecting the clinical trial standard for the clinical trial based on identifying that the clinical trial standard is the clinical standard version indicated in the configuration data.

16. A system comprising:
at least one processor; and
at least one non-transitory computer-readable storage medium storing instructions that, when executed by the at least one processor, cause the system to:
receive configuration data corresponding to a clinical trial;
select, based on the configuration data, one or more clinical trial templates from a clinical trial template library;
select, based on the configuration data, a clinical trial standard for the clinical trial from a plurality of clinical trial standards; and
generate, based on the one or more clinical trial templates and the clinical trial standard, a clinical trial database for the clinical trial by utilizing computer-executable instructions to interact with a third-party data collection system.

17. The system of claim 16, further comprising instructions that, when executed by the at least one processor, cause the system to:
identify an element by identifying elements of a graphical user interface of the third-party data collection system or identifying webpage markup of a webpage corresponding to the graphical user interface that identifies the element; and
adding template data from the one or more clinical trial templates to the element.

18. The system of claim 16, further comprising instructions that, when executed by the at least one processor, cause the system to:
identify an element of a graphical user interface of the third-party data collection system;
identifying that the element corresponds to a specific clinical trial template of the clinical trial template library; and
based on identifying that the element corresponds to the specific clinical trial template, adding data from the specific clinical trial template to the element.

19. The system of claim 16, further comprising instructions that, when executed by the at least one processor, cause the system to:
identify an element of a graphical user interface of the third-party data collection system; and
update the element by removing placeholder data of the element and adding template data or standard data to the element.

20. The system of claim 16, wherein the computer-executable instructions comprise:
instructions for identifying and interacting with elements of graphical user interfaces of the third-party data collection system;
instructions identifying template data in the one or more clinical trial templates or standard data in the clinical trial standard corresponding to the elements of the graphical user interfaces; and
instructions for interacting with the elements of the graphical user interfaces utilizing the template data or the standard data.
